(19) United States
(12) Patent Application Publication
(10) Pub. No.: US 2013/0165935 A1
(43) Pub. Date: Jun. 27, 2013

(54) EXPANDABLE RETROGRADE DRILL

(71) Applicants: Jerry R. GRIFFITHS, Pembroke, MA (US); Jose FERNANDEZ, Wrentham, MA (US)

(72) Inventors: Jerry R. GRIFFITHS, Pembroke, MA (US); Jose FERNANDEZ, Wrentham, MA (US)

(21) Appl. No.: 13/337,469

(22) Filed: Dec. 27, 2011

(51) Int. Cl. A61B 17/16

(52) U.S. Cl. 606/80

(57) ABSTRACT

An orthopedic reamer, including a first shaft; a second shaft movably disposed within at least a portion of the first shaft; a handle movably coupled to at least one of the first or second shafts; a drilling element coupled to a distal portion of the second shaft; and a plurality of cutting elements, each cutting element having a first portion connected to the first shaft and a second portion connected to the second shaft. The plurality of cutting elements are controllably transitionable from i) a substantially linear configuration parallel to the first and second shafts to ii) an angled configuration with respect to the first and second shafts.

### CROSS-REFERENCE TO RELATED APPLICATION

N/A

### STATEMENT REGARDING FEDERALLY SPONSORED RESEARCH OR DEVELOPMENT

N/A

### FIELD OF THE INVENTION

The present invention relates to instrumentation and methods of use thereof for preparing or removing tissue, and in particular, towards surgical reamers or orthopedic drilling instruments.

### BACKGROUND OF THE INVENTION

An objective of surgery in general, including orthopedic surgery, is to provide instruments, devices, and methods that are minimally invasive to the patient. Such efforts may include, for example, minimizing or reducing an incision or other access point site and/or reducing the time required for a given procedure. For drilling or reaming procedures, the size or geometry of an insertion or access incision may require sufficient dimensions to allow passage of a drill bit having a fixed width or profile. Where expansive drilled cavities are desired or necessary for placement of a prosthesis or other orthopedic hardware, the incision may, in turn, have an undesirably greater size.

Moreover, some procedures may include providing drilled cavities or regions having varying dimensions along their length. Multiple tools having differently-dimensions drill bits or the like may be utilized to provide the varied dimensions, requiring a surgeon or physician to repeat the removal and insertion of these different tools for a given procedure. Accessing the surgical site multiple times with a plurality of tools increases the duration of the surgical procedure and can increase the risks of unintended injury or consequences to the patient.

Accordingly, in view of the above, it is desirable to provide surgical drilling or reaming tools that can be used to create one or more drilling sites or cavities having varying dimensions in an efficient and minimally-invasive manner.

### SUMMARY OF THE INVENTION

The present disclosure advantageously provides methods and systems that can be used to create one or more drilling sites or cavities having varying dimensions in an efficient and minimally-invasive manner. For example, a surgical drilling instrument is provided, including an elongated first shaft defining a distal portion and a proximal portion; a drilling element coupled to the distal portion of the shaft; and a plurality of cutting elements coupled to the shaft proximal to the first drilling element, wherein the plurality of cutting elements are controllably transitionable from i) a collapsed state having a diameter substantially equal to or less than a diameter of the shaft to ii) an expanded state having a diameter greater than the diameter of the shaft. The cutting elements may be coaxial with the first shaft and/or spaced substantially equidistant from one another around a circumference of the instrument. Each cutting element may be in a substantially linear configuration in the collapsed state, may be in an angled configuration in the expanded state, and/or each cutting element may define a flexible joint. The instrument may include a second shaft movably coupled to the first shaft, where at least one of the plurality of cutting elements defines a first end coupled to the first shaft and a second end coupled to the second shaft. The plurality of cutting elements may be controllably expandable through relative movement between the first shaft and the second shaft. The instrument may include a handle coupled to the proximal portion of the first shaft, the handle being movably coupled to at least one of the first or second shafts. The instrument may include an expansion indicator on at least one of the first or second shafts.

An orthopedic reamer is disclosed, including a first shaft; a second shaft movably disposed within at least a portion of the first shaft; a handle movably coupled to at least one of the first or second shafts; a drilling element coupled to a distal portion of the second shaft; and a plurality of cutting elements, each cutting element having a first portion connected to the first shaft and a second portion connected to the second shaft. The plurality of cutting elements may be controllably transitionable from i) a substantially linear configuration parallel to the first and second shafts to ii) an angled configuration with respect to the first and second shafts. The handle may be operable to impart longitudinal movement between the first and second shafts, and/or each cutting element may be flexible.

A method of preparing a tissue site is disclosed, including positioning an orthopedic reamer in proximity to the tissue site, the reamer including a drilling element and a plurality of deployable cutting elements coupled to the drilling element; reaming a portion of the tissue site with the drilling element; deploying the plurality of cutting elements; and cutting a portion of the tissue site with the plurality of cutting elements. Each of the cutting elements may define a flexible joint. Deploying the cutting elements may include transitioning the cutting elements from a substantially linear configuration to a substantially angular configuration; transitioning the cutting elements from a first diameter substantially similar to a diameter of the drilling element to a second diameter greater than the diameter of the drilling element; and/or longitudinally displacing a first shaft of the reamer with respect to a second shaft of the reamer. The tissue site may include a femur.

### BRIEF DESCRIPTION OF THE DRAWINGS

A more complete understanding of the present invention, and the attendant advantages and features thereof, will be more readily understood by reference to the following detailed description when considered in conjunction with the accompanying drawings wherein:

FIG. 1 is an assembly view of an example of an orthopedic tissue preparation device constructed in accordance with the principles of the present disclosure;

FIG. 2 is an assembled view of the orthopedic tissue preparation device of FIG. 1 in a retracted state; and

FIG. 3 is an assembled view of the orthopedic tissue preparation device of FIG. 1 in an expanded state.

### DETAILED DESCRIPTION OF THE INVENTION

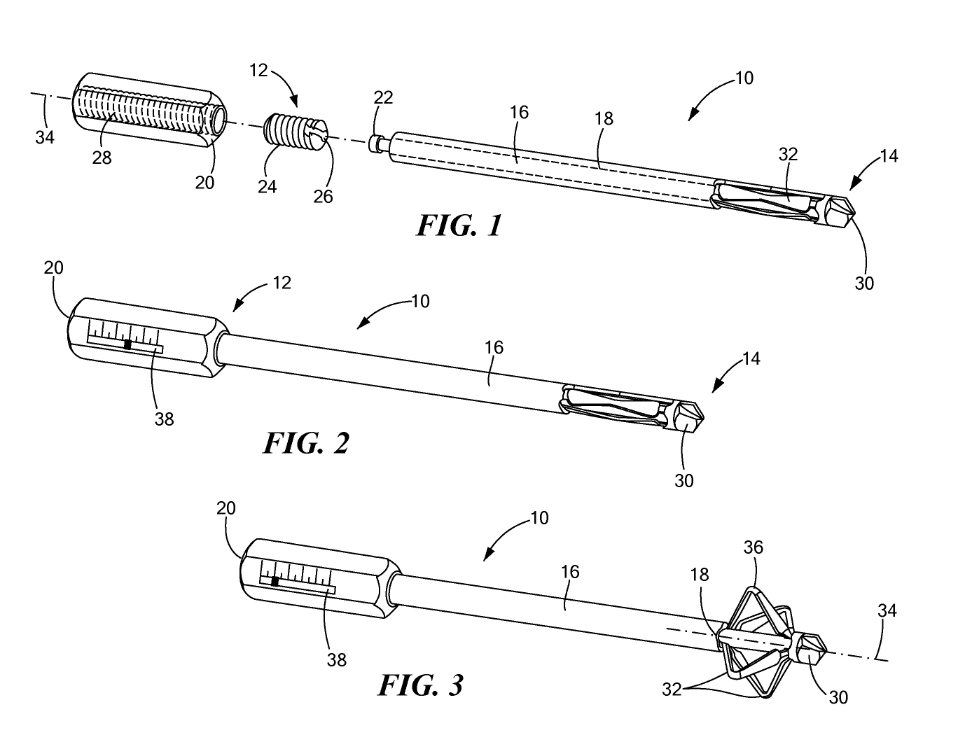

The present disclosure advantageously provides methods and systems that can be used to create one or more drilling sites or cavities having varying dimensions in an efficient and minimally-invasive manner. For example, now referring to FIGS. 1-3, a surgical or medical instrument is shown and generally designated as “10.” The instrument 10 may generally be an orthopedic drilling instrument, such as a reamer or other tissue preparation and/or removal device.

The instrument 10 generally defines a proximal portion 12 and a distal portion 14, where the proximal portion 12 is closer to or in proximity to a surgeon or user, while the distal portion 14 is closer to or in proximity to a tissue site to be treated or operated upon. The instrument 10 may include a first elongated body or shaft 16 that extends from the proximal portion 12 of the instrument 10 to the distal portion 14. The first shaft 16 may have a generally cylindrical shape and may be constructed from any of a number of inert, biocompatible materials providing sufficient rigidity for operation of the instrument 10, such as stainless steel, titanium, or other metals, polymers, and/or composites thereof for example.

The instrument 10 may also include a second elongate body or shaft 18 movably coupled to the first shaft 16. For example, the first shaft 16 may be hollow or otherwise define a passage through a portion thereof, and the second shaft 18 may be longitudinally displaceable or otherwise slidably disposed within at least a portion of the passage. The second shaft 18 may also have a generally cylindrical shape and may be constructed from any of a number of the materials described above with respect to the first shaft 16.

The first and seconds shafts may be selectively and controllably moved with respect to one another using one or more controls operable at the proximal portion of the instrument 10.

For example, the instrument 10 may include a handle 20 at the proximal portion, where manipulation of the handle 20 provides for controlled movement of the second shaft 18 relative to the first shaft 16, or vice versa. The handle 20 may generally include an elongated portion of the instrument 10 that is readily graspable by a surgeon. The handle 20 may include an enlarged diameter compared to the first and/or second shafts to both ease handling as well as impart a mechanical advantage for increased torque when turning the instrument 10. The handle 20 may include grip-enhancing features, such as one or more non-skid or increased-tactile surfaces or materials (not shown) disposed around at least a portion of the handle 20.

The handle 20 may be coupled to the first and/or second shafts to impart the controlled movement between the first and second shafts. For example, the handle 20 may be coupled to the first shaft 16 such that it is longitudinally secured to the first shaft 16, but remains rotatable around the first shaft 16. The handle 20 may further be coupled to the second shaft 18 such that the rotation of the handle 20 results in the longitudinal or slidable movement of the second shaft 18 relative to the first shaft 16. In a particular example, the second shaft 18 may include an engagement feature 22 at the proximal portion of the instrument 10. The engagement feature 22 may include a knob, protrusion, or other structure providing mechanical coupling to another component or feature of the instrument 10. The instrument 10 may include an intermediary linking element 24 that couples the handle 20 and the second shaft 18. The linking element 24 may include, for example, a threaded body having a secondary engagement feature 26 complementary to and engageable with the engagement feature 22 of the second shaft 18. For example, the linking element 24 may include a cut out or cavity that receives a protrusion or knob defining the engagement feature of the second shaft 18.

The handle 20 may define a threaded interior cavity or passage 28 that receives the linking element 24 therein, such that rotation of the handle 20 results in the proximal-distal movement of the linking element 24 within the passage 28, and thus the proximal-distal movement of the coupled second shaft 18 with respect to the first shaft 16. The handle 20 may also include a coupling for the attachment of one or more powered tools to facilitate operation of the instrument 10. For example, the instrument 10 may include a coupler or other matable feature (not shown) allowing the instrument 10 to be hydraulically, pneumatically, or electrically driven.

The instrument 10 may further include a locking element (not shown) that secures the handle 20, linking element, first shaft 16, and/or second shaft 18 in a selected position relative to one another for subsequent use of the instrument 10, as described in more detail below.

Continuing to refer to FIGS. 1-3, the instrument 10 may include a drilling element 30 at the distal portion operable to remove or displace tissue in a surgical site. The drilling element 30 may be secured to the second shaft 18 at the distal portion, for example. The drilling element 30 may include one or more drilling features or blades to facilitate its operation.

The instrument 10 may also include a plurality of cutting element 32 coupled to first and/or second shafts, where the cutting element 32 are selectively deployable or expandable to provide a range of available circumferences or diameters to remove tissue. The cutting elements 32 may be controllably expanded from a first, substantially linear configuration (such as that shown in FIG. 2) having a circumference or diameter substantially equal to or less than the diameter of the first shaft 16 and/or the drilling element 30. In this configuration, the cutting elements 32 are substantially parallel to the first and/or second shafts. This minimized, reduced profile configuration presents reduced dimensions for insertion through an incision. The cutting elements 32 may also be expanded or deployed into a plurality of second configurations having diameter(s) greater than a diameter or circumference of the first shaft 16, seconds shaft, and/or the drilling element 30 (as shown in FIG. 3). In a second configuration, the cutting elements 32 may form an angle with the first and/or second shafts or a longitudinal axis 34 thereof.

The cutting elements 32 may be sufficiently flexible to bend when transitioning from the first, reduced-profile configuration to the second, expanded configuration. For example, each cutting element may be thin enough or formed from a sufficiently elastic material to readily deform from collapsed to deployed configurations numerous times without failing. Each cutting element may define or form a joint 36 or living hinge that facilitates expansion or deployment of the cutting elements 32 through the manipulation of the first and second shafts, as described in more detail below.

The cutting elements 32 may be symmetrically disposed coaxially around the first and/or second shafts. For example, there may be four cutting elements 32 spaced equidistant from one another around a circumference of the instrument 10. Each cutting element may define one or more blade or cutting edges along a length thereof. The cutting edges or surfaces may include a scalloped edge for finer bone fragmentation when the procedure involves the removal or cutting of bone. Each cutting element may include one or more cutting surfaces or blades to allow cutting in two rotational directions (e.g., clockwise and counter-clockwise) or may be include a single cutting edge or blade such that tissue cutting or removal is limited to a single rotational direction.

The expansion or deployment of the cutting elements 32 may be achieved through the controlled longitudinal movement between the first and second shafts. For example, each cutting element may be attached or affixed to the first shaft 16 at a first end (such as towards the proximal portion 12, for example), while each cutting element may also be attached or coupled to the second shaft 18 towards the distal portion 14, for example. Referring to FIG. 2, the cutting elements 32 may be in their first, substantially linear or collapsed configuration when the second shaft 18 extends distally past the first shaft 16 to the point where the profile of the cutting elements 32 is minimized. To deploy the cutting elements 32, the second shaft 18 can be moved proximally with respect to the first shaft 16 (by turning the handle 20 and longitudinally displacing the linking element in a proximal direction, for example), as shown in FIG. 3. The movement of the second shaft 18 relative to the first shaft 16 causes the flexible cutting elements 32 to bow outward and fold or take on an angular configuration at their one or more joints.

The cutting elements 32 may be manufactured or formed through cutting the shapes or profiles of the cutting elements 32 out of an outer circumference of the first shaft 16. The cutting elements 32 may then be welded or bonded to a portion of the second shaft 18 during assembly. The cutting elements 32 accordingly provide a minimized profile for ease of insertion through a surgical incision, while retaining sufficient flexibility to deform into the radially expanded shape while also retaining sufficient rigidity to cut the desired tissue.

The instrument 10 may include an expansion indicator 38 visible to or otherwise accessible to a surgeon at the proximal portion of the device that shows information related to the expanded degree or diameter of the cutting elements 32. For example, the handle 20 may include an opening or window showing the position of a marker or the linking element 24 itself. The handle 20 may also include one or more fixed indicia indicating an expanded diameter or position of the cutting element 32 that correlate to a position of the marker or the linking element, thus allowing a surgeon to readily and confidently expand the cutting elements 32 to a desired dimension without additional imaging equipment.

In an exemplary method of use, the instrument 10 may be used to create a plurality of continuous, drilled or prepared tissue regions having varying diameters or dimensions. For example, the instrument 10 may be inserted or positioned such that the drilling element 30 is adjacent a tissue structure to be drilled or reamed, such as a femur or other orthopedic tissue site. The drilling element 30 instrument 10 may then be operated (either manually or through a powered attachment) to create a first bored or reamed passage in the tissue. The first bored or reamed region will have a diameter or circumference substantially similar to the dimensions of the drilling element 30. The instrument 10 may subsequently be positioned such that the cutting elements 32 are located adjacent to or in proximity to a second region of tissue to be treated or prepared. The cutting elements 32 may then be deployed to a desired expanded degree (as indicated on the expansion indicator or through auxiliary imaging means) to allow the creation of a second, larger bored or reamed portion having dimensions different from the first passage or portion. The deployment may be achieved through the manipulation of the relative position between the first and second shaft 18, using the handle 20 and/or linking element as described herein. The instrument 10 may be rotated or otherwise actuated during the deployment of the cutting blades for the gradual expansion and removal of tissue when transitioning from a collapsed state to an expanded, deployed state.

The cutting elements 32 may then be collapsed, repositioned, and/or deployed one or more times until the desired dimensions and characteristics of a prepared tissue region have been achieved. The cutting elements 32 may then be completely collapsed prior to removal of the device from the patient.

It will be appreciated by persons skilled in the art that the present invention is not limited to what has been particularly shown and described herein above. In addition, unless mention was made above to the contrary, it should be noted that all of the accompanying drawings are not to scale. A variety of modifications and variations are possible in light of the above teachings without departing from the scope and spirit of the invention, which is limited only by the following claims.

What is claimed is:

1. A surgical drilling instrument, comprising:
an elongated first shaft defining a distal portion and a proximal portion;
a drilling element coupled to the distal portion of the shaft; and
a plurality of cutting elements coupled to the shaft proximal to the first drilling element, wherein the plurality of cutting elements are controllably transitionable from i) a collapsed state having a diameter substantially equal to or less than a diameter of the shaft to ii) an expanded state having a diameter greater than the diameter of the shaft.

2. The instrument of claim 1, wherein the cutting elements are coaxial with the first shaft.

3. The instrument of claim 2, wherein the cutting elements are spaced substantially equidistant from one another.

4. The instrument of claim 1, wherein the each cutting element is in a substantially linear configuration in the collapsed state, and wherein each cutting element is in an angled configuration in the expanded state.

5. The instrument of claim 1, wherein each cutting element defines a flexible joint.

6. The instrument of claim 1, further comprising a second shaft movably coupled to the first shaft.

7. The instrument of claim 6, wherein at least one of the plurality of cutting elements defines a first end coupled to the first shaft and a second end coupled to the second shaft.

8. The instrument of claim 7, wherein the plurality of cutting elements are controllably expandable through relative movement between the first shaft and the second shaft.

9. The instrument of claim 1, further comprising a handle coupled to the proximal portion of the first shaft, the handle being movably coupled to at least one of the first or second shafts.

10. The instrument of claim 1, further comprising an expansion indicator on at least one of the first or second shafts.

11. An orthopedic reamer, comprising:
a first shaft;
a second shaft movably disposed within at least a portion of the first shaft;
a handle movably coupled to at least one of the first or second shafts;
a drilling element coupled to a distal portion of the second shaft; and
a plurality of cutting elements, each cutting element having a first portion connected to the first shaft and a second portion connected to the second shaft.

12. The reamer of claim 10, wherein the plurality of cutting elements are controllably transitionable from i) a substantially linear configuration parallel to the first and second shafts to ii) an angled configuration with respect to the first and second shafts.

13. The reamer of claim 10, wherein the handle is operable to impart longitudinal movement between the first and second shafts.

14. The reamer of claim 10, wherein each cutting element is flexible.

15. A method of preparing a tissue site, comprising:
positioning an orthopedic reamer in proximity to the tissue site, the reamer including a drilling element and a plurality of deployable cutting elements coupled to the drilling element;
reaming a portion of the tissue site with the drilling element;
deploying the plurality of cutting elements; and
cutting a portion of the tissue site with the plurality of cutting elements.

16. The method of claim 15, wherein each of the cutting elements defines a flexible joint.

17. The method of claim 15, wherein deploying the cutting elements includes transitioning the cutting elements from a substantially linear configuration to a substantially angular configuration.

18. The method of claim 15, wherein deploying the cutting elements includes transitioning the cutting elements from a first diameter substantially similar to a diameter of the drilling element to a second diameter greater than the diameter of the drilling element.

19. The method of claim 15, wherein deploying the cutting elements includes longitudinally displacing a first shaft of the reamer with respect to a second shaft of the reamer.

20. The method of claim 16, wherein the tissue site includes a femur.
